(19) United States
(12) Patent Application Publication
(10) Pub. No.: US 2025/0109448 A1
(43) Pub. Date: Apr. 3, 2025

(54) SIPT1 GENE REGULATING SESAME PLANT ARCHITECTURE TRAIT AND PRIMER PAIR THEREOF

(71) Applicant: HENAN SESAME RESEARCH CENTER, HENAN ACADEMY OF AGRICULTURAL SCIENCES, Zhengzhou (CN)

(72) Inventors: HONGMEI MIAO, Zhengzhou (CN); HAIYANG ZHANG, Zhengzhou (CN); MING JU, Zhengzhou (CN); CONG MU, Zhengzhou (CN); YINGHUI DUAN, Zhengzhou (CN); CHUN LI, Zhengzhou (CN); HENGCHUN CAO, Zhengzhou (CN); QIN MA, Zhengzhou (CN); GUITING LI, Zhengzhou (CN)

(21) Appl. No.: 18/978,080

(22) Filed: Dec. 12, 2024

Related U.S. Application Data
(63) Continuation of application No. PCT/CN2024/075025, filed on Jan. 31, 2024.

(30) Foreign Application Priority Data
Mar. 24, 2023 (CN) 202310296885.3

(51) Int. Cl. C12Q 1/6895; C07K 14/415; C12Q 1/6806; C12Q 1/686

(52) U.S. Cl. CPC C12Q 1/6895; C07K 14/415; C12Q 1/6806; C12Q 1/686; C12Q 2600/13

(57) ABSTRACT

A gene SiPT1 regulating a sesame plant architecture trait and a detection primer pair thereof are provided. This gene is located on 10th chromosome of sesame, belongs to a dominant control gene, has 100% explanation ratio for a branching trait; the gene has a length of 1318 bp, contains 4 exons and 3 introns, and its base sequence is shown in SEQ ID No. 1. Based on analysis and research of this gene, a good technical foundation can be laid for an early identification and rapid screening of sesame plant architecture in new varieties breeding; it has important technical significance for accelerating to breed new sesame varieties suitable for mechanized operations; can also provide a certain genetic foundation for the development of molecular assisted breeding technology and screening and breeding of new sesame varieties with different plant architectures.

### CROSS-REFERENCE TO RELATED APPLICATIONS

This application is a continuation of International Application No. PCT/CN2024/075025, filed on Jan. 31, 2024, which claims priority to Chinese Patent Application No. 202310296885.3, filed on Mar. 24, 2023, both of which are hereby incorporated by reference in their entireties.

### TECHNICAL FIELD

The present disclosure relates to the field of sesame molecular genetics and breeding technologies, and in particular, to a gene SiPT1 that regulates sesame plant architecture traits, and a primer pair thereof.

### SEQUENCE LISTING

The present application contains a sequence listing which was filed electronically in XML format and is hereby incorporated by reference in its entirety. Besides, the XML copy is created on Dec. 12, 2024, is named “SIPT1 GENE REGULATING SESAME PLANT ARCHITECTURE TRAIT AND MOLECULAR MARKER THEREOF-Sequence Listing” and is 7,388 bytes in sizes.

### BACKGROUND

Sesame (Sesamum indicum L., 2n=26) is a specific oilseed crop with high quality and plays an important role in food processing. As plant architecture influences the seed yield and cultivation management style in sesame, studying plant architecture genetics and improving plant architecture trait are an important research issue.

Statistical analysis shows that the plant architecture of most Chinese sesame varieties is uniculm. This phenotype facilitates the formation of high yield. However, the relatively high height of plantlets results in the low suitability for production mechanization. In contrast, the plant architecture of most foreign sesame varieties is branched with shorter stems and low height that are better suited for dense planting and mechanized field management, although the yield levels require improvement.

In recent years, with the advancement of biotechnological research methods, significant progress has been made in the genetics research about sesame plant architecture traits. The results indicate that sesame plant architecture trait mainly is involved in uniculm and branched phenotypes. Contrary to the uniculm trait, the branched trait is dominant and is controlled by a dominant gene pair. With the completion of the Sesame Genome Project, a strong foundation has been established for gene cloning and in-depth research of the plant architecture trait in sesame.

### SUMMARY

In the present application, 560 sesame natural germplasm accessions are used as the material basis. The gene SiPT1 regulates plant architecture in sesame was successfully cloned using natural population association analysis and other techniques. In-depth research on this gene supplies a solid technical foundation for further studies on sesame plant architecture regulation mechanism and new varieties breeding.

The technical solution of the present application is detailed as follows.

A gene SiPT1 regulating sesame plant architecture trait is located on the 10th chromosome of sesame and is a dominant control gene (contrary to the single-stem allele gene), with a 100% explanation ratio for the branched phenotype (i.e., the gene controls the branched phenotype). The gene is 1,318 bp and contains 4 exons and 3 introns, and its base sequence is shown in SEQ ID No. 1, especially, the sequence is as follows:

ATGGCAAGAATGTCAGATCCCCTCATAGTTGGAAGAGTGATAGGGGATGT
TCTTGACTCTTTCAGTCCAACTACCAAGATGTTTGTCACTTACGCTAATA
AACAAGTGTTCAATGGTCATGAGTTCTATCCTTCTGCAGTTGCTATGAAA
CCAAGGGTTGAGATTCAAGGAGGCGACTTAAGAACCTTTTACACTTTGGT
AAACACATTAATTCAATTTAATTTATCACATTTACACCACTAGATATATA
TATATATATATATATATTAACACACCTCCCGGCCCGTTCTTTTCTTTCTT
CCTTTATTTACGTGCAATAATCTATATGTCCTAACTGCTAACTCATGTTG
AAATTACATCATGTTCATGTACTGTTTTTATGTAATATTGTTGCAGGTTA
TGACTGACCCTGATGTCCCAGGCCCCAGTGATCCATATCTCAGAGAACAC
CTCCACTGGTATACATATATACAAATATACCTAGTCCGACCATGTCCAAA
ATTTATAGTTATAACCCAACGTGACGTTTATATGTAAAGCGAAACCCACT
AGGGCATTTCTGTATATATATCAGAATCAGAAATCAAAGAGATGCATACA
AGTTATATATATATATATATGTGTGTGTGTGTCTCTACCTTTAGCCCAGA
AAATGGTAAAAACTAAAGAAAACATGGGTATTCACATGTTCACTGAAACA
AGATATTTAAGCCATATCTTTATGCAGTCATCGATCTAACACGGGGATCG
ATGAGTTTACCAAGAAACTGGAAAGCTAAAGTGAGAAAGAGCTGTATGAA
TACTGAAGCTGATCAATGGGAGAATCTGCATATATATAACAATTCCAATT
CAAACCCCTAACTGACCTCAAATTTCATGGCCGGAGTACGAAGTTTTCAC
GCTCATTTATGTTACGTACTAACATTTCCTTTGGCAAACCCTATGTTACA
GGTTAGTGACCGACATCCCGGGCACCACAGATGCAACATTTGGTAAGAAC
ATCACACTGTACACACAAACACACACACACACACACGGACAAATGCGAAA
TGTGAATAACACTGCTTTTTTCCTGGTTAAAATTATTACTTCAACAGGAA
AAGAGCTGGCAAGCTACGAGATCCCGAAGCCCAACATCGGAATCCACAGG
TTTGTGTTCGTTCTCTTCAAGCAAACCGGCAGACAAACAGTAAAGAACCT
GCCTACCTCCAGAGACTGCTTCAACACCCGACGTTTCGCCGTCGAGAATG
GGCTGGGCCTCCCTGTCGCCGCCGTCTTCTTCAACGCTCAGCGCGAAACC
GCCGCCCGAAGGCGGTAG.

A cDNA corresponding to the gene SiPT1 regulating the sesame plant architecture trait is 522 bp in length and encodes 173 amino acids; specifically, the sequence is as follows:

ATGGCAAGAATGTCAGATCCCCTCATAGTTGGAAGAGTGATAGGGGATGT
TCTTGACTCTTTCAGTCCAACTACCAAGATGTTTGTCACTTACGCTAATA
AACAAGTGTTCAATGGTCATGAGTTCTATCCTTCTGCAGTTGCTATGAAA
CCAAGGGTTGAGATTCAAGGAGGCGACTTAAGAACCTTTTACACTTTGGT
TATGACTGACCCTGATGTCCCAGGCCCCAGTGATCCATATCTCAGAGAAC
ACCTCCACTGGTTAGTGACCGACATCCCGGGCACCACAGATGCAACATTT
GGAAAAGAGCTGGCAAGCTACGAGATCCCGAAGCCCAACATCGGAATCCA
CAGGTTTGTGTTCGTTCTCTTCAAGCAAACCGGCAGACAAACAGTAAAGA
ACCTGCCTACCTCCAGAGACTGCTTCAACACCCGACGTTTCGCCGTCGAG
AATGGGCTGGGCCTCCCTGTCGCCGCCGTCTTCTTCAACGCTCAGCGCGA
AACCGCCGCCCGAAGGCGGTAG.

The protein encoded by the gene SiPT1 regulating the sesame plant architecture trait contains 173 amino acids. The amino acid sequence of the protein is shown in SEQ ID NO. 2, as follows:

MARMSDPLIVGRVIGDVLDSFSPTTKMFVTYANKQVFNGHEFYPSAVAMK
PRVEIQGGDLRTFYTLVMTDPDVPGPSDPYLREHLHWLVTDIPGTTDATF
GKELASYEIPKPNIGIHRFVFVLFKQTGRQTVKNLPTSRDCFNTRRFAVE
NGLGLPVAAVFFNAQRETAARRR.

compared with branched phenotype gene SiPT1, a uniculm phenotype allele Sipt1 corresponding to the gene SiPT1 regulating the branched trait has three bases mutation in the CDS region of the gDNA sequence. At the same time, a 4,166 bp Copia-LTR transposon sequence is inserted at the 1215 bp position (this transposon sequence is unrelated to the gene sequence protected by the present application), causing pre-termination of transcription. The gene is 1,261 bp in length and contains 4 exons and 3 introns, and its base sequence is shown in SEQ ID NO. 3, as follows:

ATGGCAAGAATGTCAGATCCCCTCATAGTTGGAAGAGTGATAGGGGATGT
TCTTGACTCTTTCAGTCCAACTACCAAGATGTTTGTCACTTACGCTAATA
AACAAGTGTTCAATGGTCATGAGTTCTATCCTTCTGCAGTTGTTATGAAA
CCAAGGGTTGAGATTCAAGGAGGCGACCTAAGAACCTTTTACACTTTGGT
AAACACATTAATTCAATTTAATTTATCACATTTACACCACTAGATATATA
TATATATATATATATATTAACACACCTCCCGGCCCGTTCTTTTCTTTCTT
CCTTTATTTACGTGCAATAATCTATATGTCCTAACTGCTAACTCATGTTG
AAATTACATCATGTTCATGTACTGTTTTTATGTAATATTGTTGCAGGTTA
TGACTGACCCTGATGTCCCAGGCCCCAGTGATCCATATCTCAGAGAACAC
CTCCACTGGTATACATATATACAAATATACCTAGTCCGACCATGTCCAAA
ATTTATAGTTATAACCCAACGTGACGTTTATATGTAAAGCGAAACCCACT
AGGGCATTTCTGTATATATATCAGAATCAGAAATCAAAGAGATGCATACA
AGTTATATATATATATATATGTGTGTGTGTGTCTCTACCTTTAGCCCAGA
AAATGGTAAAAACTAAAGAAAACATGGGTATTCACATGTTCACTGAAACA
AGATATTTAAGCCATATCTTTATGCAGTCATCGATCTAACACGGGGATCG
ATGAGTTTACCAAGAAACTGGAAAGCTAAAGTGAGAAAGAGCTGTATGAA
TACTGAAGCTGATCAATGGGAGAATCTGCATATATATAACAATTCCAATT
CAAACCCCTAACTGACCTCAAATTTCATGGCCGGAGTACGAAGTTTTCAC
GCTCATTTATGTTACGTACTAACATTTCCTTTGGCAAACCCTATGTTACA
GGTTAGTGACCGACATACCGGGCACCACAGATGCAACATTTGGTAAGAAC
ATCACACTGTACACACAAACACACACACACACACACGGACAAATGCGAAA
TGTGAATAACACTGCTTTTTTCCTGGTTAAAATTATTACTTCAACAGGAA
AAGAGCTGGCAAGCTACGAGATCCCGAAGCCCAACATCGGAATCCACAGG
TTTGTGTTCGTTCTCTTCAAGCAAACCGGCAGACAAACAGTAAAGAACCT
GCCTACCTCCAGAGATGTTAGGAAATGGATCGGGTTAAGATGGATAACAG
GTGGAATATGA.

The cDNA corresponding to the uniculm phenotype allele Sipt1 is 465 bp in length and encodes 154 amino acids; specifically:

ATGGCAAGAATGTCAGATCCCCTCATAGTTGGAAGAGTGATAGGGGATGT
TCTTGACTCTTTCAGTCCAACTACCAAGATGTTTGTCACTTACGCTAATA
AACAAGTGTTCAATGGTCATGAGTTCTATCCTTCTGCAGTTGTTATGAAA
CCAAGGGTTGAGATTCAAGGAGGCGACCTAAGAACCTTTTACACTTTGGT
TATGACTGACCCTGATGTCCCAGGCCCCAGTGATCCATATCTCAGAGAAC
ACCTCCACTGGTTAGTGACCGACATACCGGGCACCACAGATGCAACATTT
GGAAAAGAGCTGGCAAGCTACGAGATCCCGAAGCCCAACATCGGAATCCA
CAGGTTTGTGTTCGTTCTCTTCAAGCAAACCGGCAGACAAACAGTAAAGA
ACCTGCCTACCTCCAGAGATGTTAGGAAATGGATCGGGTTAAGATGGATA
ACAGGTGGAATATGA;

compared with the cDNA corresponding to branched phenotype gene SiPT1 and the protein encoded by the branched phenotype gene SiPT1, the 48th amino acid valine (V) encoded by the cDNA sequence of the uniculm phenotype gene is mutated to alanine (A). Additionally, transcription pre-termination forms for the mutation of amino acid sequences from the 123th to 154th amino acids. The change results in the alteration of protein domain and ultimately causes the shift of plant architecture from branched to uniculm phenotype.

A protein encoded by the uniculm phenotype allele Sipt1 or its corresponding protein contains 154 amino acids, and its amino acid sequence is shown in SEQ ID No. 4; as follows:

MARMSDPLIVGRVIGDVLDSFSPTTKMFVTYANKQVFNGHEFYPSAVVMK
PRVEIQGGDLRTFYTLVMTDPDVPGPSDPYLREHLHWLVTDIPGTTDATF
GKELASYEIPKPNIGIHRFVFVLFKQTGRQTVKNLPTSRDVRKWIGLRWI
TGGI.

A primer pair for obtaining the gene SiPT1 regulating the sesame plant architecture trait or its corresponding uniculm phenotype allele Sipt1 by PCR amplification, the primer pair is designed as follows:

a positive Primer PT1 Primer F:
5′-ATGGCAAGAATGTCAGATCCCC-3′;
a reverse Primer PT1 Primer R:
5′-CCGCCTTCGGGCGGCGGT-3′.

A PCR amplification method for obtaining the gene SiPT1 regulating the sesame plant architecture trait or its corresponding uniculm phenotype allele Sipt1 with the primer pair for PCR amplification, includes the following steps:
(1) preparing a template for PCR amplification
extracting genomic DNA from sesame germplasm accessions with branched phenotype or uniculm phenotype;
sesame germplasm accession with branched phenotype is Yinni Heli;
sesame germplasm accession with uniculm phenotype is Yuzhi 11;
(2) PCR amplification
taking the genomic DNA extracted in step (1) as a template, performing PCR amplification using the PCR primer pair (PT1 Primer F/R); the reference amplification for a 25 μL reaction system is as follows:
Template DNA (50 ng/μL), 1.0 μL;
2×Prime STAR Max Premix, 12.5 μL;
Forward Primer 1 (10 μM), 1.0 μL;
Reverse Primer (10 μM), 1.0 μL;
ddH2O was added to 25 μL;
a specific PCR amplification reference procedure is as follows: pre-denaturation at 98° C. for 3 minutes; denaturation at 98° C. for 15 seconds, renaturation at 58° C. for 15 seconds, extension at 72° C. for 4 minutes, and 35 cycles; followed by a final extension at 72° C. for 5 minutes;
in the amplification product, the amplicon length of the obtained gene SiPT1 regulating the sesame plant architecture trait is 1,318 bp; the amplicon length of the obtained uniculm phenotype allele Sipt1 containing a transposon sequence is 4,166 bp.

A primer pair for detecting the gene SiPT1 regulating the sesame plant architecture trait or its corresponding uniculm phenotype allele Sipt1, where a PCR amplification method is used to detect and distinguish whether a sample contains the gene SiPT1 regulating the sesame plant architecture trait or its corresponding uniculm phenotype allele Sipt1;
during designing the primer pair SiPT1 F1/R1, the primer sequences of SiPT1 F1 for both the branching phenotype (SiPT1 gene) and the uniculm phenotype (Sipt1 allele) are identical; the primer sequence of SiPT1 R1 is designed according to the retrotransposon sequence of the uniculm phenotype;
the primer pair is designed as follows:

SiPT1 F1:
5′-TACAGGTTAGTGACCGACAT-3′,
SiPT1 R1:
5′-TGAGACGAGGGTGAGTGT-3′;

during PCR amplification, no amplification product is observed in the electrophoresis result for the branched phenotype sample (SiPT1 gene), whereas the amplification product for the uniculm phenotype sample (allele Sipt1) is 491 bp in length;
the sequence of the amplification result (491 bp) is as follows:

TGAGACGAGGGTGAGTGTTGTCTTTCACGTGTGGTGAAGACTTGCATACA
AGTGTGTGTGTGTTGTACTCCTCAATTTTCCTCCTCTTTGAGTATTTTCT
CCTGGCTTGGTATCGCCCCCAGACGTAGGATTTATATCCAAACTGGGTTA
ACAAATTCGTGTGTCTTTTATCGCCTTCATATTCCACCTGTTATCCATCT
TAACCCGATCCATTTCCTAACATCTCTGGAGGTAGGCAGGTTCTTTACTG
TTTGTCTGCCGGTTTGCTTGAAGAGAACGAACACAAACCTGTGGATTCCG
ATGTTGGGCTTCGGGATCTCGTAGCTTGCCAGCTCTTTTCCTGTTGAAGT
AATAATTTTAACCAGGAAAAAAGCAGTGTTATTCACATTTCGCATTTGTC
CGTGTGTGTGTGTGTGTGTTTGTGTGTACAGTGTGATGTTCTTACCAAAT
GTTGCATCTGTGGTGCCCGGTATGTCGGTCACTAACCTGTA.

A method for detecting and determining the sesame plant architecture phenotype using the primer pair includes the following steps:
(1) sample preparation
extracting genomic DNA from the test sesame germplasm samples;
(2) PCR amplification
using the genomic DNA extracted in step (1) as a template, perform PCR amplification with primer pair (SiPT1 F1/R1) for detection;
conducting electrophoresis or sequencing analysis on the PCR amplification product;
(3) result determination
determining the phenotype based on the electrophoresis and/or sequencing analysis results from step (2); the specific criterion for determination are as follows:
if there is no amplification band in the electrophoresis result after amplification, the tested germplasm accession is homozygous and dominant with branched phenotype;
if there is one amplification band (with a length of 491 bp) in the PCR product or the sequencing result is 491 bp, the tested germplasm accession is either homozygous and recessive with single stem phenotype, or heterozygous with branched phenotype.

Application of the gene SiPT1 regulating the sesame plant architecture trait in new variety breeding. This gene is associated with the sesame architecture phenotype and is used for breeding new crop varieties with branched phenotype (example for sesame).

Considering the important role of plant architecture phenotype in sesame yield formation and mechanization production, the inventors conducted further genetics analysis of plant architecture trait in sesame. During the genome-wide association analysis (GWAS) of plant architecture trait using 560 sesame natural germplasm accessions, statistical results showed that the branch number in the samples ranges from 1 to 4 branches (in 2017 environment) and from 1 to 6 branches (in 2018 environment). GWAS results determined that the most significantly associated SNP locus for the branching trait was at 3453283 bp (p=6.58E-20) on 10th chromosome of the sesame genome. The highest explanation ratio of the locus for trait variation was 17%. Haplotype block analysis further indicated that there were no other tightly associated SNP sites near this locus. The locus is located in gene Sindi_2199200, which was further named the sesame branching gene SiPT1. Furthermore, a structural analysis of the target gene was performed using Yuzhi 11 (uniculm phenotype) and the representative germplasm accession “Yinni Heli” (branching phenotype). The analysis confirmed that there was a sequence deletion in the uniculm phenotype samples.

Overall, combining with sesame genome fine mapping and resequencing technologies, the branching gene SiPT1 regulating sesame plant architecture trait has been successfully cloned and obtained in the present application. The main technical achievements of this application can be summarized as follows:
(1) the gene location and related primer pair design based on the branching gene SiPT1 provide a solid technical foundation for early identification and rapid screening of sesame plant architecture phenotypes in new varieties breeding;
(2) As plant architecture (branching) trait has the significant impact on sesame production including yield and planting style, this gene has substantial technical significance for accelerating new sesame varieties breeding suitable for mechanized production;
(3) the branching gene SiPT1 and its corresponding detection method can provide a foundation for analyzing the regulation mechanisms of plant architecture and other traits in sesame and other crops, and supply the genetic basis for the development of sesame molecular-assisted breeding technologies, screening and breeding new sesame varieties with diverse plant architecture traits, and enhancing the sesame breeding efficiency and genetics and breeding level. Therefore, this work possesses the considerable scientific research value and economic application potential.

### BRIEF DESCRIPTION OF DRAWINGS

FIG. 1 shows a field image comparison of the uniculm phenotype (left image, Yuzhi 11) and the branching phenotype (right image, Yinni Heli) involved in the present application.

FIG. 2 shows a Mahattan plot of genome-wide association analysis of plant architecture phenotypes in relevant sesame natural populations. The upper image displays the Mahattan plot for the GWAS analysis of plant architecture phenotypes in 2017 year environment; while the lower image shows the Mahattan plot for the GWAS analysis in 2018 year environment.

FIG. 3 presents a schematic diagram illustrating the differences in sequence alignment of the sesame branching gene SiPT1 and its alleles;
in color view: the green box represents exons, the black horizontal line represents introns, and the blue box and line segment denote inserted LTR retrotransposon sequence of Copia class (Copia_LTR_retrotransposon). The arrow indicates nucleotide sequence difference points between the SiPT1 gene and the allele Sipt1.

FIG. 4 displays a comparative result of the protein sequences encoded by the sesame branching gene SiPT1 and its alleles.

FIG. 5 shows the PCR amplification results of the sesame branching gene primer pair SiTP1 F1/R1 in some sesame germplasm accessions;

lane M is DL 500 marker, and the displayed bands from top to bottom are 500 bp, 400 bp, 300 bp, 200 bp, 150 bp, 100 bp, and 50 bp, respectively;
lane 1-12 represent 12 uniculm sesame germplasm accessions (CX038, CX075, CX080, CX125, CX011, CX020, CX037, CX049, CX060, CX062, CX071, and Yuzhi No. 11);
lane 13-23 represent 11 branching sesame germplasm accessions (CX174, CX073, CX118, CX119, CX304, CX305, CX334, CX336, CX343, CX457, and CX462);
lane 24 represents a wild specie S. radiatum (G02, branching phenotype).

### DESCRIPTION OF EMBODIMENTS

A further explanation of the present application is provided in combination with the Examples below. Before introducing the specific Examples, a brief introduction and explanation of certain experimental backgrounds information related to the following Examples are presented.

### Biological Materials

Sesame variety Yuzhi 11 is a common and widely used material in sesame breeding and cultivation, it belongs to a uniculm (phenotype) variety;

Yinni Heli is sesame germplasm accession with branching phenotype. The main characteristics includes branching, four capsule ridges, and brown seeds. This germplasm accession is reserved in the sesame germplasm resource bank of the Henan Sesame Research Center, Henan Academy of Agricultural Sciences.

Other sesame germplasm accessions (such as CX038, CX075, CX080, CX125, CX011, CX020, CX037, CX049, CX060, CX062, CX071, X174, CX073, CX118, CX119, CX304, CX305, CX334, CX336, CX343, CX457, and CX462) also come from the germplasm resource bank of Henan Sesame Research Center, Henan Academy of Agricultural Sciences;

it should be noted that the use and study of the related germplasm materials comply with applicable administrative regulations. These germplasm materials can be obtained from the public accessible germplasm bank of Henan Sesame Research Center, the Henan Academy of Agricultural Sciences or other public germplasm resource libraries.

### Example 1

Considering the important role of sesame plant architecture phenotype in sesame yield and mechanized varieties breeding, combined with the planting results of relevant natural populations, the inventors first conducted a statistical analysis of the phenotypic traits of sesame plant architectures. Additionally, a whole-genome association analysis of related phenotypic traits was performed. Related information is briefly introduced as follows.

### I. Investigation and Statistics of Stem Phenotype (Branching or Uniculm)

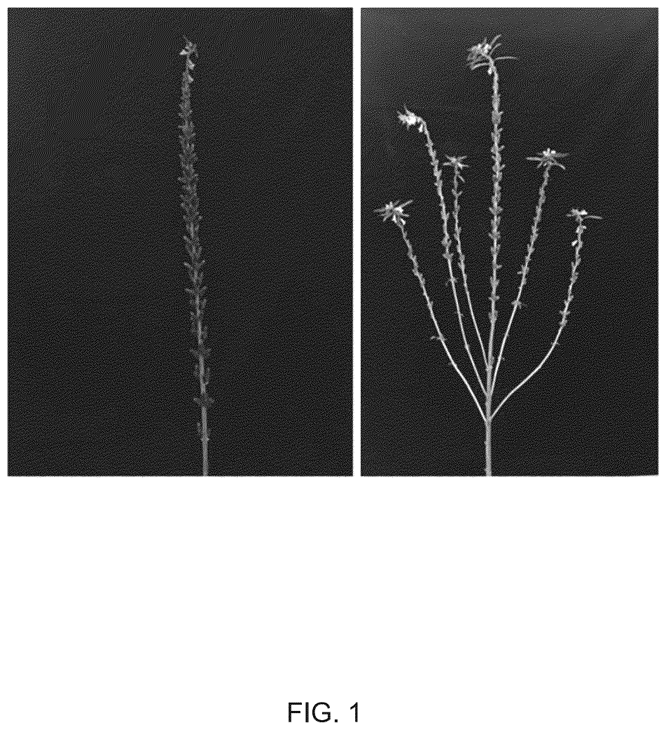

From 2017 to 2018, 560 natural populations of sesame with different phenotypes were planted and investigated. Comparison of branching and uniculm phenotype in sesame germplasm accessions is shown in FIG. 1. The specific statistical results are presented in Table 1.

TABLE 1
Statistical result of plant architecture phenotype in
sesame germplasm materials

| | | Branching | Uniculm | Other |
|---|---|---|---|---|
| | Sample | phenotype (plant | phenotype | phenotype |
| Year | number | number) | (plant number) | (plant number) |
| 2017 | 560 | 382 | 178 | 0 |
| 2018 | 560 | 348 | 199 | 13 |

“* others” refer to the samples that plant phenotype data have not been obtained in the field.

It should be noted that during the investigation and statistics, the branching phenotype refers to the sample with 2 or more stems with capsules, while the uniculm phenotype refers to the sample with 1 stem with capsules.

The above statistical results indicate that the branching phenotype of sesame is a dominant trait, contrary to the uniculm phenotype, and controlled by a gene pair. Additionally, the statistical results for the branching and the uniculm phenotypes under two years environments for the 560 germplasm accessions mentioned above show that the ratios of branching phenotype to uniculm phenotype are 2.15:1 and 1.75:1, respectively, which are suitable for subsequent genetics analysis.

### II. Genome Wide Association Analysis of Sesame Plant Architecture Trait

### (1) Genome Sequencing

for the 560 germplasm-population mentioned above, the genomic DNA were extracted using the CTAB method (refers to: Sesame DNA and RNA simultaneous extraction method, 2008, Molecular Plant Breeding). Genome resequencing of the 560 materials was performed using the Illumina sequencing method, with a sequencing coverage of ≥10×.

### (2) Data Comparison and Splicing

With the genome data of Yuzhi 11 (Zhang et al., Ultra-dense SNP genetic map construction and identification of SiDt gene controlling the determinate growth habit in Sesamum indicum L, 2016, Science Reports; Zhao et al., Identification of sesame (Sesame indicum L.) chromosomes using the BAC-FISH system, 2018, Plant Biology) as refence genome, the resequencing data of each line in step (1) was aligned and assembled using BWA (Burrows Wheeler Aligner) software.

### (3) Genome-Wide Association Analysis

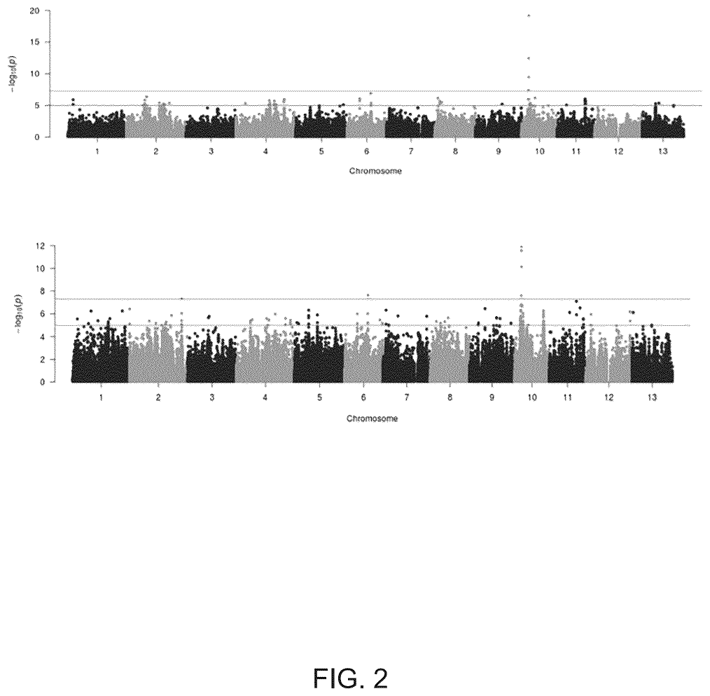

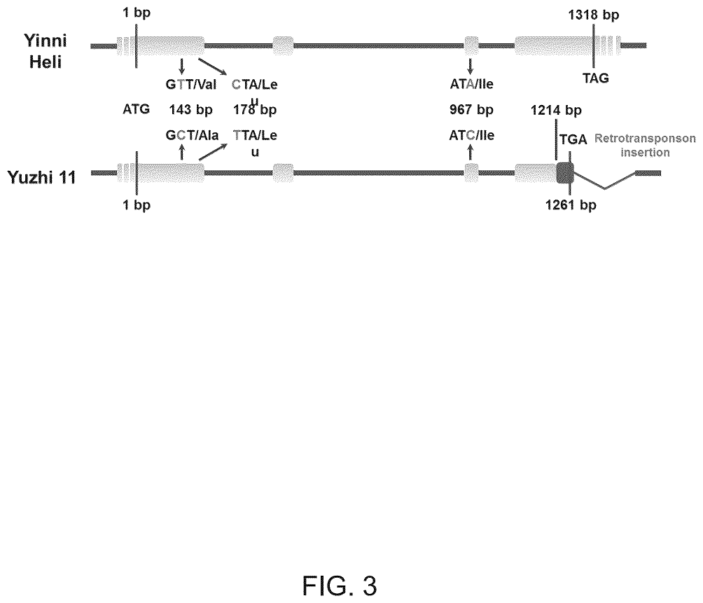

Based on the aforementioned work, SNP matrix analysis was further conducted. After extracting the vcf (Variant Call Format) files of 560 sesame germplasm accessions, a total of 4,008,867 variants were identified. After preliminary filtered, 896,745 high-quality variants retained. Subsequently, these data were converted into a format suitable for Tassele 5.0 software. Upon completion of the conversion, in order to identify variants and P-values that are closely associated with the sesame plant architecture trait, a mixed linear model (MLM) correlation analysis was performed using the phenotype data in 2017 and 2018. Partial results are shown in FIG. 2. The analysis results revealed that the SNP locus with the smallest P-value closely associated to the target trait was located on chromosome 10: SNP3453283, P=6.58E-20 (2017 phenotype association results). This locus also is the same SNP locus with the smallest P-value (P=7.34E-11) closely linked to the target trait in 2018.

A statistical analysis of all the variants in the 200 kb upstream and downstream of the SNP locus was conducted, with a parameter set as follows: minimum minor allele frequency (minMAF): 0.05; maximum LD comparison distance: 100 kb. Haplotype analysis indicated that this region contains a total of 99 SNP/InDel polymorphic loci. Of which 5 loci are located within gene sequences. The specific results are shown in Table 2.

TABLE 2
Information on candidate variants closely
associated with the sesame branching trait

| Chromosomal | | | Located | Gene | Gene | Gene |
|---|---|---|---|---|---|---|
| location | SNP no. | P value * | gene no. | start site | end site | orientation |
| Sichr. 10 | 3339449 | 0.00348 | 2197500 | 3335668 | 3339681 | + |
| Sichr. 10 | 3443478 | 0.00184 | 2198900 | 3439039 | 3443450 | − |
| Sichr. 10 | 3453283 | 7.34E−11 | 2199200 | 3452165 | 3453425 | − |
| Sichr. 10 | 3459618 | 0.2834 | 2199300 | 3458802 | 3461581 | + |
| Sichr. 10 | 3493164 | 0.08794 | 2200100 | 3492098 | 3497202 | − |

note:
the data is the association result of 2018 phenotype data.

Further analysis revealed that there are no other significantly associated variants within the region containing the target SNP3453283. This result indicates that the gene Sindi_2199200 with SNP3453283 is likely the target gene. This gene is designated as SiPT1.

### Example 2

Based on the localization of plant architecture regulation gene in Example 1, further cloning and sequencing analysis of the target gene were performed. The specific process is briefly introduced as follows.

### (1) Preparation of DNA Template

DNA from the branched phenotype “Yinni Heli” and the uniculm phenotype Yuzhi 11 was used as templates, respectively (specific extraction refers to Example 1).

### (2) Primer Pair Design

Based the associated variant and target gene obtained in Example 1, primer pairs for PCR amplification were designed according to the sesame reference genome (Yuzhi 11, uniculm phenotype) data as follows:

a Positive Primer TP1 Primer F:
5′-ATGGCAAGAATGTCAGATCCCC-3′,
a Reverse Primer TP1 Primer R:
5′-CCGCCTTCGGGCGGCGGT-3′.

### (3) PCR Amplification

with the DNA of the branching phenotype “Yinni Heli” and the uniculm phenotype Yuzhi 11 as templates, PCR amplification was performed using the PT1 Primer F/R primer pair and Takara high-fidelity enzyme R045Q. The reference setup for the 25 μL reaction system is as follows:
Template DNA (50 ng/μL), 1.0 μL;
2×PrimeSTAR Max Premix, 12.5 μL;
Forward Primer 1 (10 μM), 1.0 μL;
Reverse Primer (10 μM), 1.0 μL;
adding ddH₂O to 25 μL;

the specific amplification reference procedure (PTC-100 thermal cycler, a product of MJ Research company) is as follows: pre-denaturation at 98° C. for 3 minutes; denaturation at 98° C. for 15 seconds, denaturation at 58° C. for 15 seconds, extension at 72° C. for 4 minutes, with a total of 35 cycles; finally, extension at 72° C. for 5 minutes. The amplified product can be stored at 4° C. or directly subjected to electrophoresis detection and analysis.

Electrophoresis detection was performed on the amplification product, which was then recovered for sequencing (the related primer sequence synthesis and amplicon sequencing work in the present application were completed by Tianjin Gene Chip Biotechnology Co., Ltd.). A comparative analysis of the sequencing results (as illustrated in the sequence comparison diagram in FIG. 3) indicates that:
the branching gene SiPT1 regulating the plant architecture trait in the branching phenotype “Yinni Heli” is 1,318 bp (sequence shown in SEQ ID No. 1) and contains 4 exons and 3 introns;
the uniculm phenotype allele Sipt1 in the uniculm phenotype Yuzhi 11 has a 3-base mutation in the coding sequence (CDS) region of gDNA, compared with the branching phenotype gene SiPT1. Additionally, an insertion of 4,166 bp Copia_LTR transposon sequence is detected at the 1,215 bp locus, which results in the premature termination of gene transcription. This allele gene is 1,261 bp (sequence shown in SEQ ID NO. 3) and also contains 4 exons and 3 introns.

Furthermore, specific differences between the SiPT1 gene and its allele Sipt1 include: a T/C mutation at the 143 base, a C/T mutation at the 178 base, and an A/C mutation at the 967 base in the CDS region. The allele Sipt1 has a Copia_LTR_retrotransposon insertion at the 1214 base, leading to a termination codon (TGA) at the 1261 base;

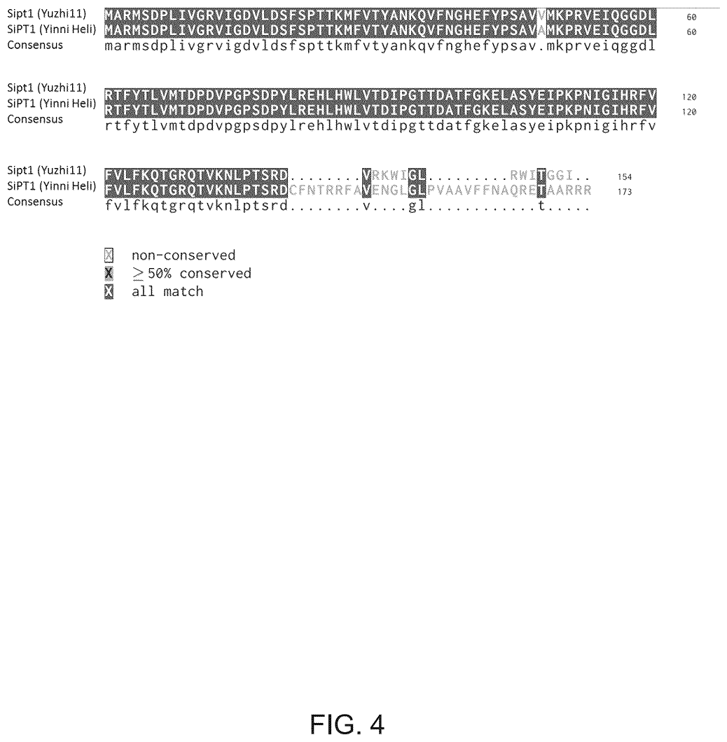

due to these sequence differences, the encoded proteins exhibit the following changes:
(1) the 48th amino acid was mutated from valine (V) to alanine (A);
(2) the amino acids from 123 bp to 154 bp underwent a sequence change, resulting in premature termination of translation (as shown in FIG. 4).

Based on the above results, it can be preliminarily concluded that the insertion of the Copia_LTR-retrotransposon caused a structural change in the sesame PT1 protein, ultimately leading to a change in plant architecture phenotype from branching to single stem.

### Example 3

On the basis of the above results, in order to further confirm that the SiPT1 gene regulates the branching trait of the sesame plant architecture phenotype, the inventors designed the detection primer pairs. One hundred branching/uniculm sesame germplasm accessions, the wild specie S. radiatum (branching phenotype), and an F₂ population derived from a combination of Yinni Heli and Yuzhi 11 were randomly chosen and used for validation. The specific process is briefly described as follows.

### (1) Primer Pair Design

Based on the sequencing analysis results mentioned above, a primer pair for PCR amplification detection was designed. The design principle considered that the forward primer is identical in both the branching phenotype (SiPT1 gene) and the uniculm phenotype (Sipt1 allele). The reverse primer was designed according to a retrotransposon sequence of the uniculm phenotype, favoring for detection and differentiation of PCR amplification. The primer pair is designed as follows:

SiPT1F1:
5′-TACAGGTTAGTGACCGACAT-3′,
SiPT1R1:
5′-TGAGACGAGGGTGAGTGT-3′.

### (2) Preparing a Template

Young leaves of 100 branching/uniculm sesame germplasm accessions (randomly chosen from the 560 germplasm accessions described in Example 1), the wild specie S. radiatum (branching phenotype), and individual plants of F₂ population derived from Yinni Heli×Yuzhi 11 as sample sources were used to extract DNA as PCR templates. The plant architecture trait of each sample was confirmed in advance. The heterozygous or homozygous genotype of the individual F₂ plantlets in the combination of Yinni Heli and Yuzhi 11 was determined based on the phenotypes of their F₂₃ line. Genomic DNAs were extracted to serve as templates for PCR amplification.

### (3) PCR Amplification

Using the DNAs prepared in step (2) as templates, PCR amplification was performed with the primer pair SiPT1 F1/R1 designed in step (1). The reference settings for 25 μL reaction system during a PCR amplification are as follows:
Template DNA (50 ng/μL), 1.0 μL;
2×PrimeSTAR Max Premix, 12.5 μL;
Forward Primer 1 (10 μM), 1.0 μL;
Reverse Primer (10 μM), 1.0 μL;
adding ddH₂O to 25 μL;

a reference for PCR reaction program (using the PTC-100 thermal cycler, a product of MJ Research company) is as follows: pre-denaturation at 98° C. for 3 minutes, denaturation at 95° C. for 15 seconds, renaturation at 58° C. for 15 seconds, extension at 72° C. for 15 seconds, repeating this cycle 35 times, and finally, extension at 72° C. for 5 minutes. The amplified product can be stored at 4° C. or directly subjected to electrophoresis detection (using a gel concentration of 1%, at a voltage of 150 V, for 20 minutes) for analysis.

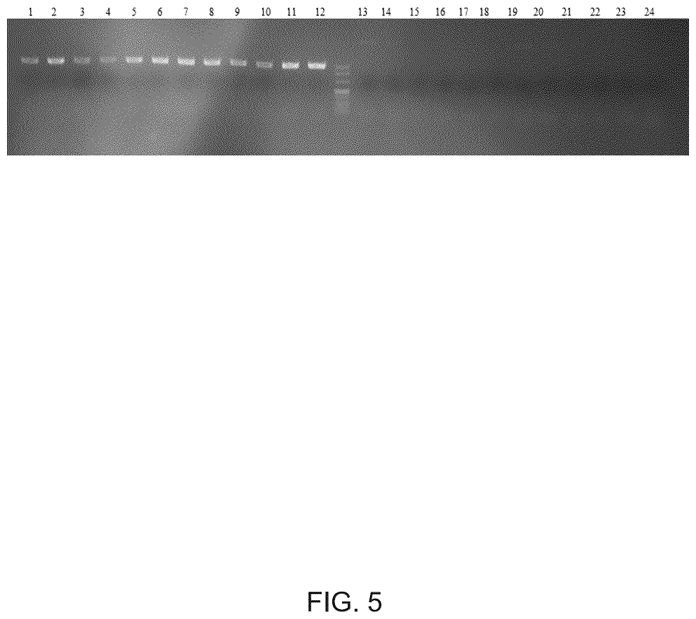

Part of the electrophoresis results is shown in FIG. 5. The analysis indicates that:
a plant with branching phenotype has no amplification band;
a homozygous parent with uniculm phenotype has a target band of 491 bp;
a heterozygous plant has a band of 491 bp after amplification, while the plant architecture belongs to the branching phenotype.

In summary, it can be concluded that the SiPT1 gene is a key gene responsible for sesame plant architecture change. The designed primer pair is allowed for accurate detection of this gene and facilitates early determination of sesame plant architecture traits (branching or uniculm type). Further in-depth research in this gene has significant technical implication for understanding the regulation mechanism of plant architecture trait in sesame and other crops, enabling molecular marker-assisted selection and breeding new sesame varieties with different plant architecture phenotypes.

What is claimed is:

1. A gene, SiPT1, regulating a sesame plant architecture trait, is characterized by being located on the 10th chromosome of sesame. It is classified as a dominant control gene and has a 100% explanation ratio for the branching trait. The gene has a length of 1,318 bp, contains 4 exons and 3 introns, and its nucleotide sequence is shown in SEQ ID No. 1;
the cDNA corresponding to the gene SiPT1, which regulates the sesame plant architecture trait, has a sequence length of 522 bp and encodes 173 amino acids, as follows:
ATGGCAAGAATGTCAGATCCCCTCATAGTTGGAAGAGTGATAGGGGATGT
TCTTGACTCTTTCAGTCCAACTACCAAGATGTTTGTCACTTACGCTAATA
AACAAGTGTTCAATGGTCATGAGTTCTATCCTTCTGCAGTTGCTATGAAA
CCAAGGGTTGAGATTCAAGGAGGCGACTTAAGAACCTTTTACACTTTGGT
TATGACTGACCCTGATGTCCCAGGCCCCAGTGATCCATATCTCAGAGAAC
ACCTCCACTGGTTAGTGACCGACATCCCGGGCACCACAGATGCAACATTT
GGAAAAGAGCTGGCAAGCTACGAGATCCCGAAGCCCAACATCGGAATCCA
CAGGTTTGTGTTCGTTCTCTTCAAGCAAACCGGCAGACAAACAGTAAAGA
ACCTGCCTACCTCCAGAGACTGCTTCAACACCCGACGTTTCGCCGTCGAG
AATGGGCTGGGCCTCCCTGTCGCCGCCGTCTTCTTCAACGCTCAGCGCGA
AACCGCCGCCCGAAGGCGGTAG.

2. A protein encoded by the gene SiPT1 regulating the sesame plant architecture trait or its corresponding cDNA according to claim 1, characterized by containing 173 amino acids, wherein the amino acid sequence is shown in SEQ ID NO. 2.

3. An uniculm phenotype allele Sipt1 corresponding to the gene SiPT1 regulating the sesame plant architecture trait according to claim 1, characterized by having a length of 1,261 bp, contains 4 exons and 3 introns, and wherein its nucleotide sequence is shown in SEQ ID NO. 3;
the cDNA corresponding to the uniculm phenotype allele Sipt1 has a sequence length of 465 bp and encodes 154 amino acids, as follows:
ATGGCAAGAATGTCAGATCCCCTCATAGTTGGAAGAGTGATAGGGGATGT
TCTTGACTCTTTCAGTCCAACTACCAAGATGTTTGTCACTTACGCTAATA
AACAAGTGTTCAATGGTCATGAGTTCTATCCTTCTGCAGTTGTTATGAAA
CCAAGGGTTGAGATTCAAGGAGGCGACCTAAGAACCTTTTACACTTTGGT
TATGACTGACCCTGATGTCCCAGGCCCCAGTGATCCATATCTCAGAGAAC
ACCTCCACTGGTTAGTGACCGACATACCGGGCACCACAGATGCAACATTT
GGAAAAGAGCTGGCAAGCTACGAGATCCCGAAGCCCAACATCGGAATCCA
CAGGTTTGTGTTCGTTCTCTTCAAGCAAACCGGCAGACAAACAGTAAAGA
ACCTGCCTACCTCCAGAGATGTTAGGAAATGGATCGGGTTAAGATGGATA
ACAGGTGGAATATGA.

4. A protein encoded by the uniculm phenotype allele Sipt1 or its corresponding cDNA according to claim 3, characterized by containing 154 amino acids, wherein the amino acid sequence is shown in SEQ ID No. 4.

5. A primer pair for PCR amplification to obtain the gene SiPT1 regulating the sesame plant architecture trait according to claim 1 or its corresponding uniculm phenotype allele Sipt1, wherein the primer pair is designed as follows:
a positive Primer PT1 Primer F:
5′-ATGGCAAGAATGTCAGATCCCC-3′;
a reverse Primer PT1 Primer R:
5′-CCGCCTTCGGGCGGCGGT-3′.

6. A PCR amplification method for obtaining the gene SiPT1 regulating sesame plant architecture traits or its corresponding uniculm phenotype allele Sipt1 using the primer pair according to claim 5, comprising the following steps:
(1) preparing a template for PCR amplification
extracting a genomic DNA from sesame germplasm accessions with branching phenotype or uniculm phenotype;
wherein the branching phenotype sesame germplasm is Yinni Heli;
and the uniculm phenotype sesame germplasm is Yuzhi 11;
(2) PCR amplification
using the genomic DNA extracted in step (1) as a template, performing PCR amplification with the primer pair;
an amplification product corresponding to the gene SiPT1 regulating sesame plant architecture traits has a length of 1,318 bp.

7. A primer pair for detecting the gene SiPT1 regulating the sesame plant architecture traits or its corresponding uniculm phenotype allele Sipt1 according to claim 1, characterized in that it is used in a PCR amplification method to detect and distinguish whether a sample contains the gene SiPT1 or its corresponding uniculm phenotype allele Sipt1;
the primer pair is designed as follows:
SiPT1 F1:
5′-TACAGGTTAGTGACCGACAT-3′,
SiPT1 R1:
5′-TGAGACGAGGGTGAGTGT-3′.

8. A method for detecting and determining the plant architecture phenotype of sesame using the detection primer pair according to claim 7, comprises the following steps:
(1) sample preparation
extract genomic DNA from the sesame germplasm of the sample to be tested;
(2) PCR amplification
using the genomic DNA extracted in step (1) as a template, perform PCR amplification with the detection primer pair SiPT1 F1/R1;
then subject the PCR amplification product to electrophoresis or sequencing analysis;
(3) Result determination
determine the result based on the electrophoresis and/or sequencing result in step (2), with the following criteria:
if there is no amplification band in the electrophoresis result, it indicates that the sesame germplasm is dominant homozygous with a branching phenotype;
if there is only one amplification band in the product, or the sequencing result shows 491 bp, it indicates that the sesame germplasm is either recessive homozygous with a uniculm phenotype, or heterozygous with a branching phenotype.

9. An application of the gene SiPT1 regulating the sesame plant architecture trait according to claim 1 in plant variety breeding, wherein the gene is associated with the branching phenotype of sesame and is used for breeding new varieties with the branching phenotype.


SEQUENCE LISTING (4 sequences)

SEQ ID NO: 1 - DNA, 1318 bp, Sesamum indicum L.
  source 1..1318: mol_type = genomic DNA
atggcaagaatgtcagatcccctcatagttggaagagtgataggggatgttcttgactctttcagtccaactaccaagatgtttgtcacttacgctaataaacaagtgttcaatggtcatgagttctatccttctgcagttgctatgaaaccaagggttgagattcaaggaggcgacttaagaaccttttacactttggtaaacacattaattcaatttaatttatcacatttacaccactagatatatatatatatatatatatattaacacacctcccggcccgttcttttctttcttcctttatttacgtgcaataatctatatgtcctaactgctaactcatgttgaaattacatcatgttcatgtactgtttttatgtaatattgttgcaggttatgactgaccctgatgtcccaggccccagtgatccatatctcagagaacacctccactggtatacatatatacaaatatacctagtccgaccatgtccaaaatttatagttataacccaacgtgacgtttatatgtaaagcgaaacccactagggcatttctgtatatatatcagaatcagaaatcaaagagatgcatacaagttatatatatatatatatgtgtgtgtgtgtctctacctttagcccagaaaatggtaaaaactaaagaaaacatgggtattcacatgttcactgaaacaagatatttaagccatatctttatgcagtcatcgatctaacacggggatcgatgagtttaccaagaaactggaaagctaaagtgagaaagagctgtatgaatactgaagctgatcaatgggagaatctgcatatatataacaattccaattcaaacccctaactgacctcaaatttcatggccggagtacgaagttttcacgctcatttatgttacgtactaacatttcctttggcaaaccctatgttacaggttagtgaccgacatcccgggcaccacagatgcaacatttggtaagaacatcacactgtacacacaaacacacacacacacacacggacaaatgcgaaatgtgaataacactgcttttttcctggttaaaattattacttcaacaggaaaagagctggcaagctacgagatcccgaagcccaacatcggaatccacaggtttgtgttcgttctcttcaagcaaaccggcagacaaacagtaaagaacctgcctacctccagagactgcttcaacacccgacgtttcgccgtcgagaatgggctgggcctccctgtcgccgccgtcttcttcaacgctcagcgcgaaaccgccgcccgaaggcggtag

SEQ ID NO: 2 - protein, 173 aa, Sesamum indicum L.
MARMSDPLIVGRVIGDVLDSFSPTTKMFVTYANKQVFNGHEFYPSAVAMKPRVEIQGGDLRTFYTLVMTDPDVPGPSDPYLREHLHWLVTDIPGTTDATFGKELASYEIPKPNIGIHRFVFVLFKQTGRQTVKNLPTSRDCFNTRRFAVENGLGLPVAAVFFNAQRETAARRR

SEQ ID NO: 3 - DNA, 1261 bp, Sesamum indicum L.
  source 1..1261: mol_type = genomic DNA
atggcaagaatgtcagatcccctcatagttggaagagtgataggggatgttcttgactctttcagtccaactaccaagatgtttgtcacttacgctaataaacaagtgttcaatggtcatgagttctatccttctgcagttgttatgaaaccaagggttgagattcaaggaggcgacctaagaaccttttacactttggtaaacacattaattcaatttaatttatcacatttacaccactagatatatatatatatatatatatattaacacacctcccggcccgttcttttctttcttcctttatttacgtgcaataatctatatgtcctaactgctaactcatgttgaaattacatcatgttcatgtactgtttttatgtaatattgttgcaggttatgactgaccctgatgtcccaggccccagtgatccatatctcagagaacacctccactggtatacatatatacaaatatacctagtccgaccatgtccaaaatttatagttataacccaacgtgacgtttatatgtaaagcgaaacccactagggcatttctgtatatatatcagaatcagaaatcaaagagatgcatacaagttatatatatatatatatgtgtgtgtgtgtctctacctttagcccagaaaatggtaaaaactaaagaaaacatgggtattcacatgttcactgaaacaagatatttaagccatatctttatgcagtcatcgatctaacacggggatcgatgagtttaccaagaaactggaaagctaaagtgagaaagagctgtatgaatactgaagctgatcaatgggagaatctgcatatatataacaattccaattcaaacccctaactgacctcaaatttcatggccggagtacgaagttttcacgctcatttatgttacgtactaacatttcctttggcaaaccctatgttacaggttagtgaccgacataccgggcaccacagatgcaacatttggtaagaacatcacactgtacacacaaacacacacacacacacacggacaaatgcgaaatgtgaataacactgcttttttcctggttaaaattattacttcaacaggaaaagagctggcaagctacgagatcccgaagcccaacatcggaatccacaggtttgtgttcgttctcttcaagcaaaccggcagacaaacagtaaagaacctgcctacctccagagatgttaggaaatggatcgggttaagatggataacaggtggaatatga

SEQ ID NO: 4 - protein, 154 aa, Sesamum indicum L.
MARMSDPLIVGRVIGDVLDSFSPTTKMFVTYANKQVFNGHEFYPSAVVMKPRVEIQGGDLRTFYTLVMTDPDVPGPSDPYLREHLHWLVTDIPGTTDATFGKELASYEIPKPNIGIHRFVFVLFKQTGRQTVKNLPTSRDVRKWIGLRWITGGI